(19) United States
(12) Patent Application Publication
(10) Pub. No.: US 2024/0156420 A1
(43) Pub. Date: May 16, 2024

(54) DUAL-LAYER DETECTOR SYSTEM AND METHOD FOR SPECTRAL IMAGING AND CONTRAST ENHANCED DIGITAL BREAST TOMOSYNTHESIS

(71) Applicant: THE RESEARCH FOUNDATION FOR THE STATE UNIVERSITY OF NEW YORK, Albany, NY (US)

(72) Inventors: Anthony R. LUBINSKY, Port Jefferson, NY (US); Wei ZHAO, East Setauket, NY (US); Xiaoyu DUAN, Stony Brook, NY (US); Hailiang HUANG, Stony Brook, NY (US); Adrian HOWANSKY, Centereach, NY (US); Jann STAVRO, Stony Brook, NY (US); Amirhossein GOLDAN, Stony Brook, NY (US)

(73) Assignee: THE RESEARCH FOUNDATION FOR THE STATE UNIVERSITY OF NEW YORK, Albany, NY (US)

(21) Appl. No.: 18/280,001

(22) PCT Filed: Mar. 1, 2022
(86) PCT No.: PCT/US2022/018255

§ 371 Date: Sep. 1, 2023

Related U.S. Application Data
(60) Provisional application No. 63/154,879, filed on Mar. 1, 2021.

(51) Int. Cl. A61B 6/02; A61B 6/00; A61B 6/40; A61B 6/42; A61B 6/50

(52) U.S. Cl. CPC A61B 6/025; A61B 6/4042; A61B 6/4241; A61B 6/481; A61B 6/482; A61B 6/502

(57) ABSTRACT

Structures and methods operable to detect radiation are described. The structure includes a dual-layer detector imaging device that permits one-shot of x-rays for dual-energy imaging. In one embodiment, a front layer of the detector includes a photon counting detector and aback layer of the detector includes an an x-ray radiation source for absorbing x-ray radiation to separate radiation into low energy and high energy components for incidence upon an imaging object. In an embodiment, the imaging object includes a contrast agent material having a characteristic K-edge atomic energy band level, and the separation filter absorbing the X-ray radiation is near the K-edge atomic energy band level.

### CROSS REFERENCE TO RELATED APPLICATIONS

This application claims the benefit of U.S. Provisional Application No. 63/154,879 filed on Mar. 1, 2021, the entirety of which is incorporated by reference.

### FIELD

The present application relates generally to radiation detectors and digital radiography and 3D digital breast imaging.

### BACKGROUND

In digital radiography, an imaging system may include a Flat Panel Detector (FPD) including a collection of layers such as a scintillator screen that absorbs radiation and produces pulses of visible light upon x-ray absorption, a pixelated array of photosensors, e.g., photodiodes, (where the produced light is sensed) and a thin film transistor array. to generate electrical signals. The generated electrical signals may be used by the imaging system to produce a digital image. In some examples, a quality (e.g., sharpness, resolution) of the produced image may be affected by various phenomenon such as light scattering, and/or other phenomena.

Spectral x-ray imaging extracts material-specific images of a volume of interest. This technique is used in clinical radiography to provide additional diagnostic information about patient anatomy. Spectral imaging is performed by acquiring two or more x-ray transmission images (i.e. “projections”) of a volume of interest, each with a different x-ray energy, and applying post-processing techniques to identify its constituent materials by differences in their x-ray attenuation properties. One strategy to achieve energy separation between images is by temporal subtraction, wherein an x-ray source kVp (kilovoltage peak) and/or filter is changed between successive x-ray projections

Improving the contrast or signal-to-noise ratio (SNR) of material-selective x-ray images by using high energy and low energy projection measurements is described in Alvarez, U.S. Pat. No. 4,029,963. U.S. Pat. No. 4,445,226 to Brody discloses a method eliminating soft tissue or bone structure from an x-ray image using a hybrid energy subtraction technique. U.S. Pat. No. 8,792,617 to Baetz discloses a method to create dual energy x-ray images in mammography using two exposures, with differing kVp and filtration for each. However, this method is subject to problems and artifacts due to the motion of the patient between the two exposures.

FIG. 1B depicts a prior art approach 10 for dual-energy subtraction, post contrast injection, or temporal subtraction strategies before and after contrast injection that have been employed in dual-shot/single detector spectral mammography systems and spectral computed tomography (CT) systems. This approach uses a fast kV-switching to produce alternating high and low-energy projections. For instance, in the dual-shot method approach of FIG. 1B, a first filter 20 is located proximately in front of X-ray radiation source 15 for passing only low energy X-ray radiation 25 for incidence upon the imaging object 12. A single detector 30, such as an x-ray photoconductor or scintillating screen, receives the photons from the low energy X-ray radiation 25 that is transmitted through and not absorbed by the object 12 and associated circuitry converts these detected photons into electrical signals used to produce a first low energy (LE) image of the object. Subsequently, in the dual-shot method approach of FIG. 1B, a second filter 40 is located proximately in front of X-ray radiation source 15 for passing only high energy X-ray radiation 25 for incidence upon the imaging object 12. The single detector 30 receives the photons from the high energy X-ray radiation 45 that is transmitted through and not absorbed by the object 12 and associated circuitry converts these detected photons into electrical signals used to produce a first high energy (HE) image of the object. Dual-shot methods that take two exposures may achieve a high degree of spectral separation between high and low energy images, because they permit changes in x-ray source kVp, filter and image receptor in the acquisition workflow. Although this flexibility is desirable for achieving high contrast spectral imaging, this approach is intrinsically limited by misregistration artifacts that arise from subject motion between acquisitions.

Alternatively, U.S. Pat. No. 9,526,466 to Karim discloses a method to create dual energy x-ray images in mammography using a stacked integrating multilayer detector in which the detectors are exposed to the x-ray beam in the same x-ray exposure at the same time. However, this method suffers from limited energy separation between the low and high energy images created, and resultant loss of contrast and SNR in the energy subtracted image, due to the lack of a spectral separation filter, as in the present disclosure.

Finally, U.S. Pat. No. 7,342,233 to Danielsson claims an apparatus with an array of photon counting channels where each channel converts individual x-ray detection events into electrical pulses according to pulse height, and where each of two sets of counters count pulses according to whether they are higher or lower than a given threshold to create high energy and low energy images, in a single exposure.

### SUMMARY

Accordingly, disclosed are structures, imaging systems and detectors that provide improved image quality and dose performance. The system and imaging method includes a single-layer or dual-layer detector that permits one-shot (single exposure) of x-rays for dual-energy imaging, i.e., a single shot energy discrimination,

In an example embodiment, the system including a single-layer or dual-layer detector comprises a spectral separation filter located proximate the output of an X-ray energy source for modulating the X-ray radiation into low energy and high energy bands for permitting one-shot of x-rays for dual-energy imaging of objects.

In an embodiment, the imaging object can include a contrast agent material having a characteristic K-edge atomic energy band level. The separation filter is of a material that absorbs X-ray radiation near that K-edge atomic energy band level to create two bands of X-ray radiation: a low energy (LE) radiation band and a high energy (HE) radiation band for the dual-energy imaging of objects.

The apparatus may further comprise a single-layer X-ray energy image detector including a front X-ray imaging photon-counting detector (PCD) for receiving incident X-ray radiation transmitted through the spectral separation filter and imaging object and generating electrical signals capable of producing a first LE image of the imaging object and generating further electrical signals capable of producing a second HE image of the imaging object.

In an embodiment, the PCD is an amorphous-Selenium (α-Se) x-ray photon counting flat-panel imager (SWAD) for detecting both lower energy and higher energy photons to form a respective low energy image and high energy image.

Further, the apparatus may comprise the spectral separation filter and a dual-layer X-ray energy image detector including a first front direct-conversion x-ray imaging detector located on an underlying substrate for producing LE and HE images of the object and a second back indirect-conversion x-ray imaging detector underlying the substrate for producing information to be combined with the HE image of the object.

In this embodiment, the first front X-ray imaging detector can comprise the PCD, such as an amorphous-Selenium (α-Se) x-ray photon counting flat-panel imager (SWAD) for detecting both lower energy and higher energy photons that is used to form a respective low energy image and high energy image. The second back X-ray imaging detector includes an indirect-conversion flat panel x-ray detector or photon energy integrating detector and comprises a material of an atomic number chosen to efficiently detect higher energy photons to form a high energy image that is used to enhance the higher energy image obtained by the first front PCD.

In yet a further embodiment of the dual-layer X-ray imaging detector, both the first front X-ray imaging detector and second back X-ray imaging detector comprises an integrating detector. The front integrating detector comprises first pixel sensors for directly converting first energy level band photons of the incident radiation transmitted through the imaging object into first image signals configurable to form a low energy image of the imaging object. The back integrating detector formed below the substrate comprises second pixel sensors for converting second energy level band photons of the incident radiation transmitted through the imaging object and through the front integrating detector and the substrate and into second image signals configurable to form a high energy image of the imaging object.

In accordance with a first aspect of the invention, there is provided an apparatus comprising: a separation filter for spectrally separating radiation from an X-ray radiation source into a first energy level band and a second energy level band for incident radiation upon an imaging object; a substrate; an x-ray photon counting detector formed on the substrate, the x-ray photon counting detector comprising an array of detector pixels, each detector pixel comprising a sensor for detecting interactions of individual x-ray photons of the incident radiation transmitted through the imaging object during a fixed period of time; and each detector pixel of the array having an associated count circuit operable to generate a first electrical signal representing a respective count of the number of detected interactions of individual x-ray photons of the first energy level band and a second electrical signal representing a respective count of the number of detected interactions of individual x-ray photons of the second energy level band, wherein the first electrical signals and second electrical signals from the detector pixels of the array provide respective energy spectral images of the imaging object.

In a further aspect, an apparatus comprises: a separation filter for spectrally separating radiation from an X-ray radiation source into first energy level band and second energy level band for incident radiation upon an imaging object; a first substrate; an x-ray photon counting front detector formed on the first substrate, the x-ray photon counting detector comprising an array of detector pixels, each detector pixel comprising a sensor for detecting interactions of individual x-ray photons of the incident radiation transmitted through the imaging object during a fixed period of time; each detector pixel of the array having an associated count circuit operable to generate a first electrical signal representing a respective count of the number of detected interactions of individual x-ray photons of the first energy level band and a second electrical signal representing a respective count of the number of detected interactions of individual x-ray photons of the second energy level band, wherein the first electrical signals and second electrical signals from the detector pixels of the array provide respective energy spectral images of the imaging object; and a back detector formed on a second substrate and located below the first substrate, the back detector comprising: a scintillating screen for converting incident radiation containing x-ray photons of the second energy level band transmitted through the imaging object and through the front detector into light photons; and a photosensor array disposed between the scintillating screen and the second substrate for the back detector, the photosensor array operable to capture the light photons from the scintillating screen and convert the captured light photons into further electrical signals, the further electrical signals operable for combination with the second electrical signals from the front detector pixel array to obtain images of the imaging object.

In a further aspect, an apparatus comprises: a separation filter for spectrally separating radiation from an X-ray radiation source into a first energy level band and second energy level band for incident radiation upon an imaging object, the second energy level band being of a greater energy than the first energy level band; a glass substrate; a front integrating detector formed on the glass substrate, the front integrating detector comprising first pixel sensors for directly converting first energy level band x-ray photons of the incident radiation transmitted through the imaging into first image signals configurable to form a low energy image of the imaging object; and a back integrating detector formed below the glass substrate, the back integrating detector comprising second pixel sensors for indirectly converting second energy level band x-ray photons of the incident radiation transmitted through the imaging object and through the front integrating detector and the substrate and into second image signals configurable to form a high energy image of the imaging object.

Further to this aspect, the front integrating detector comprises: a first photoconductive layer for converting incident radiation containing x-ray photons of the first energy level band transmitted through the imaging object into a charge; and a first charge storage array disposed between the first photoconductive layer and the substrate for storing charges associated with the converted x-ray photons.

Further to this aspect, the back integrating detector comprises: a scintillating screen for converting incident radiation containing x-ray photons of the second energy level band transmitted through the imaging object into light photons; and a photosensor array disposed between the second scintillating screen and the substrate, the photosensor array operable to capture the light photons from the second scintillating screen and convert the captured light photons into the second imaging signals.

Further to this aspect, the imaging object includes a contrast agent material having a characteristic K-edge atomic energy band level, the separation filter having an x-ray absorption edge for absorbing the X-ray radiation near, e.g., within 10 keV of, the K-edge atomic energy band level of the contrast agent material.

Further features as well as the structure and operation of various embodiments are described in detail below with reference to the accompanying drawings. In the drawings, like reference numbers indicate identical or functionally similar elements.

### BRIEF DESCRIPTION OF THE DRAWINGS

FIG. 1A illustrates an example dual-layer detector imaging system that permits one-shot of x-rays for dual-energy imaging in an aspect of the disclosure.

FIG. 1B depicts a standard single detector system that requires two-shots of x-ray exposures for dual-energy imaging;

FIGS. 2A-2E illustrate example plots depicting a comparison of HE and LE spectral separation for dual-shot/single detector and dual-layer detector approaches.

FIG. 2F depicts a table summarizing example mean LE and mean HE spectral energy band values obtained for each of the embodiments of FIGS. 2A-2E;

FIGS. 3A-3C illustrates the impact of filter thickness and thickness of the top detector material (T1) on spectral separation achieved for the dual-detector spectral imaging approach depicted embodiment of FIG. 1A; FIG. 3D is a table summarizing the resulting improvements in image quality as measured by a SDNR for different thicknesses of a front detector layer and back detector layers for the single shot/dual layer detector of FIG. 1A;

FIG. 4 shows a first embodiment of a spectral imaging system including a single shot/single layer detector for producing dual LE and HE spectral images of an object;

FIG. 5 depicts a cross-sectional schematic of a fully assembled SWAD device for use as a front detector in a single-detector or dual-detector spectral imaging approach according an embodiment;

FIG. 6 depicts example results of a simulated pixel response of a photon counting SWAD device such as depicted in FIG. 5;

FIG. 7 depicts a second embodiment of a spectral imaging system including a single shot/dual layer detector for producing dual LE and HE spectral images of an object;

FIG. 8 depicts a further embodiment of a spectral imaging system including a single shot/dual layer detector for producing dual LE and HE spectral images of an object;

FIG. 9 illustrates a further embodiment of a spectral imaging system including a single shot/dual layer detector including a direct-conversion x-ray front x-ray detector layer and an indirect-conversion back x-ray detector layer x-ray detector;

FIG. 10 illustrates a further embodiment of a spectral imaging system including a dual shot exposure/dual layer detector either with a source array or step-and-shoot tomography system in an aspect of the disclosure;

FIGS. 11A-11E depict simulation result images of a digital mammography, contrast enhanced dual energy mammography, digital breast tomosynthesis and contrast enhanced digital breast tomosynthesis;

FIG. 12 illustrates a table depicting the Signal-Difference-to-Noise-Ratio in the simulated contrast enhanced dual energy mammography results of FIGS. 11C, 11D and 11E; and

FIGS. 13A-13C depict respective LE DBT image slice, HE DBT image and CE DBT image slices illustrating the issues to be overcome with prior art dual-shot imaging approaches.

### DETAILED DESCRIPTION

The following detailed description of aspect of the disclosure will be made in reference to the accompanying drawings. In this disclosure, explanation about related functions or constructions known in the art are omitted for the sake of clearness in understanding the concept of the disclosure to avoid obscuring the disclosure with unnecessary detail.

Spectral X-ray imaging extracts material-specific images of a volume of interest. This technique is used in clinical radiography to provide additional diagnostic information about patient anatomy. Spectral imaging is performed by acquiring two or more x-ray transmission images (i.e. “projections”) of a volume of interest, each with a different x-ray energy, and applying post-processing techniques to identify its constituent materials by differences in their x-ray attenuation properties.

In embodiments herein, there are two strategies to achieve energy separation between images: 1. temporal subtraction, wherein the x-ray source kVp and/or filter is changed between successive x-ray projections, or 2. single-shot energy discrimination, wherein multiple detectors are provided that are sensitive to different energy spectra. In an embodiment herein, single shot energy discrimination is performed using a single photon counting detector (PCD) where two or more energy bins are used to form multiple energy-specific images from a single x-ray projection. In a further embodiment, single shot energy discrimination is performed using a dual detector including a photon counting detector and a photon energy integrating detector (EID) used to form multiple energy-specific images from a single x-ray projection. A further embodiment implements single shot energy discrimination using a dual detector including two photon EIDs to form multiple energy-specific images from a single x-ray projection. A further embodiment contemplates a dual detector to form multiple energy-specific images using two or more radiation exposure (multiple shots). The two strategies can be combined to further enhance energy discrimination for spectral imaging.

Spectral imaging using single-shot energy discrimination is free from motion misregistration artifacts and, as further described herein, can be used for spectral DBT applications to offer a more complete and accurate diagnostic information about the breast compared to conventional 2D full-field digital mammography (FFDM), spectral FFDM, and conventional DBT. This information includes, 3D breast tissue density, 3D microcalcification distribution and type, 3D distribution of contrast agent (e.g. iodine), and 3D material decomposition of breast lesions of interest (e.g. mass). The present disclosure describes a spectral imaging system implementation that may be used, for example, to provide one or all the above information in spectral DBT.

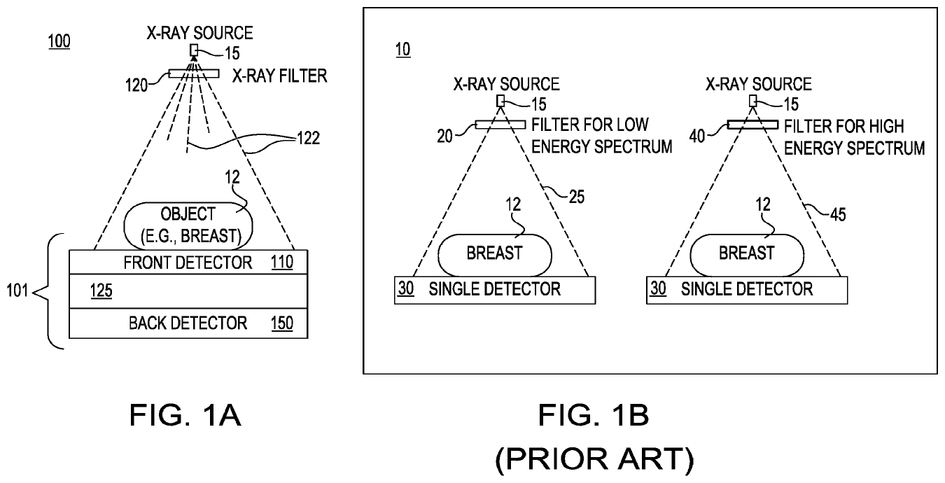

FIG. 1A illustrates one embodiment of a dual-layer detector spectral imaging system 100 that permits one-shot of x-rays for dual-energy imaging of an object 12.

In an embodiment, the spectral imaging system 100 includes an X-ray source 15 which may be a X-ray tube that produces X-rays, or other devices that may produce X-rays. The X-ray source may irradiate X-ray radiation through a spectral separation filter onto the subject, where the subject may absorb a portion of the X-rays, causing an attenuation of the X-rays. The attenuated X-rays may be directed towards the dual-detector structure 101 as incident X-rays 122.

In either embodiment, the dual layer X-ray spectral imaging detector approach includes an X-ray filter 120 that is located proximately in front of an X-ray radiation source 15 for absorbing a portion of X-ray radiation output of the radiation source 15 such that x-ray radiation 122 is simultaneously passed at two energy levels, a first LE energy level that includes a radiation band of energy below the separation filter's x-ray absorption edge and a second HE energy level that includes a radiation band of energy above the separation filter's x-ray absorption edge. The system 100 includes a dual layer X-ray imaging detector for receiving incident x-ray radiation 122 transmitted through the object 12. The dual layer X-ray imaging detector in configured as a stack 101 including a first front detector 110 disposed on a substrate 125 and a second back detector 150 located or attached underneath the substrate 125 using an radiotransparent adhesive, for example. In an embodiment, the front detector 110 can include a photon counting detector (PCD) or a photoconductor type of photon integrating detector. The back detector 150 can include a light photon integrating detector. In these embodiments, the substrate can include a glass or like material substrate 125.

In some of the embodiments herein, the spectral separation filter 120 at the x-ray source output, includes one or more materials with atomic numbers Z_{F1} to Z_{FN} and thicknesses T_{F1} to T_{FN}, to modulate the x-ray energy spectrum incident on the imaging subject 12. Filter materials are chosen with atomic numbers that selectively remove x-ray energies from the beam via preferential attenuation at energies near their K-edges, e.g., within 10 keV. Filter materials, thicknesses, and the order of their arrangement with respect to the x-ray source are chosen to shape the filter's energy transmission characteristics according to the initial energy spectrum, detector properties and spectral information of interest. For example, materials with K-edges near (e.g., within 10 keV) that of the breast imaging contrast material (e.g., iodine) can be selected. Example separation filter materials can include Rh, Ag, Pd, In and Sn filters.

In an embodiment, the dual-layer detector imaging system 100 is particularly configured for digital breast tomosynthesis (DBT) and particularly for obtaining high-resolution 3-dimensional (3D) X-ray images of a breast 12. In such an embodiment, the first front detector 110 is a direct-conversion flat panel x-ray detector (“front detector”) that is first exposed to the x-ray beam transmitted through the imaging subject, having a lower atomic number, e.g., atomic number Z₁ and thickness T₁, and high spatial resolution. The front detector material's atomic number and thickness are chosen to preferentially absorb lower energy x-rays while transmitting higher energies, thereby allowing formation of a low energy image. Its high spatial resolution is leveraged to preserve image detail information, i.e., small structures and sharp edges. In an example, front detector 110 is an amorphous selenium detector (e.g., Z₁=34, T₁=150 μm or 200 μm).

In an embodiment, the second back detector 150 is an indirect-conversion flat panel x-ray detector (“back detector”) having atomic number Z₃>Z₁ and thickness T₃. The back detector's atomic number is chosen to efficiently detect higher energy photons to form a high energy image. The back detector's atomic number may be matched to the K-edge of a contrast agent, e.g. CsI:Tl for iodine, to improve the agent's conspicuity in high energy images. The back detector can include a scintillator that may be transparent or optically-turbid, structured or unstructured (e.g., columnar Cs:Tl or powder Gd₂O₂S:Tb), and comprise optically reflective or absorptive backings. The scintillator is coupled to a photodetector array by direct deposition, pressure contact, or in some embodiments by using a fiber optic plate to transmit the x-ray-induced light image to the photodetector without light spreading. The light sensors may be α-Si:H photodiodes, MIS-type, or other types known in the art. The thin film transistor (TFT) switching elements may be the α-Si:H type, a metal oxide (MOTFT) types, or other types known in the art.

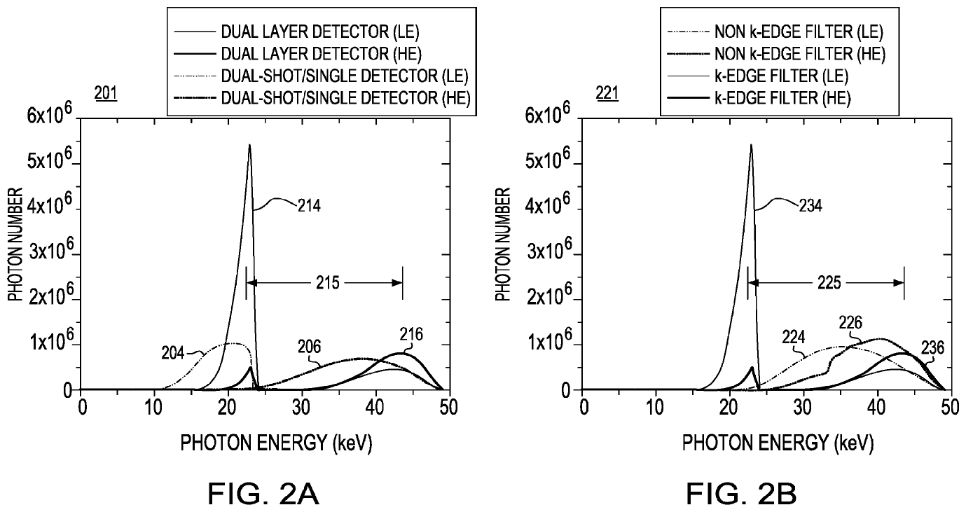

FIG. 2A-2E illustrate respective plots depicting the ability of the dual-layer approach 100 of FIG. 1A for spectrally separating the x-ray energy spectrum into LE and HE spectral energy bands for incidence upon the imaging object. For example, each of the plots of FIGS. 2A-2D show the spectral separation of the x-ray radiation and particularly exemplary plots depicting the number of photons received in the filtered radiation forming LE and HE spectral imaging bands as a function of photon energies (in keV) of the filtered radiation as achieved by the dual layer detector approach 100 of FIG. 1A for W/Rh spectrum. Comparative plots of the spectral separation of the x-ray radiation and particularly exemplary plots depicting the number of photons received in the filtered radiation as a function of photon energies (in keV) of the filtered radiation as achieved by the dual-shot/single detector approach 10 of FIG. 1B is additionally shown. In the example plots shown in FIGS. 2A-2D, the detector is a 150 μm thick amorphous selenium (α-Se) energy integrating detector (EID) and the spectral separation filter comprises Rhodium at 200 μm thick.

In a first depiction, FIG. 2A shows a comparison 201 of the spectral separation achieved for the single shot/dual layer detector approach of FIG. 1A as compared to the dual-shot/single layer detector approach of FIG. 1B. As shown in FIG. 2A, the single shot/dual layer detector approach detects LE spectral radiation energy 214 that far surpasses the LE spectral radiation energy 204 detected by the dual-shot/single layer detector approach of FIG. 1B. Similarly, as shown in the plot of FIG. 2A, the single shot/dual layer detector approach detects HE spectral radiation energy 216 that surpasses the HE spectral radiation energy 206 as detected by the dual-shot/single layer detector approach of FIG. 1B. Additionally, the LE and HE radiation achieves a widened separation 215 of approximately 20 keV resulting in improved LE and HE images.

FIG. 2B shows a comparison 221 of the spectral separation achieved for the single shot/dual layer detector approach of FIG. 1A using a k-edge band spectral separation filter for single shot LE/HE radiation separation as compared to the single layer detector approach of FIG. 1B that uses a non-k-edge filter for LE and HE spectral separation. As shown in FIG. 2B, the single shot/dual layer detector approach using a k-edge band filter detects LE spectral radiation energy 234 and HE spectral energy 236 having respective spectral energy peaks separation 225 of about 20 keV that far surpasses the spectral separation achieved by dual-shot/single layer detector and a non k-edge filtering approach of FIG. 1B which is shown in FIG. 2B as the LE spectral radiation energy 224 and HE spectral radiation energy 226 having little or no spectral energy separation.

FIG. 2C shows a plot 241 of the spectral separation achieved for the single shot/dual layer detector approach of FIG. 1A using a front α-Se photoconductive detector 110, a back CsI energy integrating scintillating detector 150, and a k-edge band spectral separation filter for single shot LE/HE radiation separation. As shown in FIG. 2C, the single shot/dual layer detector approach using a front α-Se photon integrating detector 110, a back photon integrating detector 150 and a k-edge band filter detects LE spectral radiation energy 244 and HE spectral energy 246. Enhanced spectral energy peak separation 245 of greater than 20 keV is achieved between the LE and HE bands resulting in improved LE and HE images.

FIG. 2D shows a plot 261 of the spectral separation achieved for the single shot/dual layer detector approach of FIG. 1A using a front α-Se photoconductive detector 110, a back α-Se photoconductive detector 150, and a k-edge band spectral separation filter for single shot LE/HE radiation separation. As shown in FIG. 2D, the single shot/dual layer detector approach using a front α-Se photon integrating detector 110 a back α-Se photon integrating detector 150 and a k-edge band filter detects LE spectral radiation energy 254 and HE spectral energy 256. Enhanced spectral energy peak separation 255 of greater than 20 keV is achieved between the LE and HE bands resulting in improved LE and HE images.

FIG. 2E shows a plot 281 of the spectral separation achieved for the single shot/dual layer detector approach of FIG. 1A using a front CsI energy integrating scintillating detector 110, a back CsI energy integrating scintillating detector 150, and a k-edge band spectral separation filter for single shot LE/HE radiation separation. As shown in FIG. 2E, the single shot/dual layer detector approach using a front CsI photon integrating detector 110 a back CsI photon integrating detector 150 and a k-edge band filter detects large amounts of LE spectral radiation energy 274 and HE spectral energy 276.

FIG. 2F is a table 290 depicting a column 292 providing a mean value of the detected LE spectral band energy in keV and a column 294 providing a mean value of the detected HE spectral band energy in keV for the embodiments of FIGS. 2A-2E. As shown in FIG. 2F, while the mean LE and mean HE energy spectral separation of about 17 keV is achieved for a dual-shot/single layer detector, the dual layer detector configurations of FIG. 1A using the k-edge band filtering and having the front α-Se photoconductive detector 110 achieve comparable mean LE and mean HE band separation of about 13 keV.

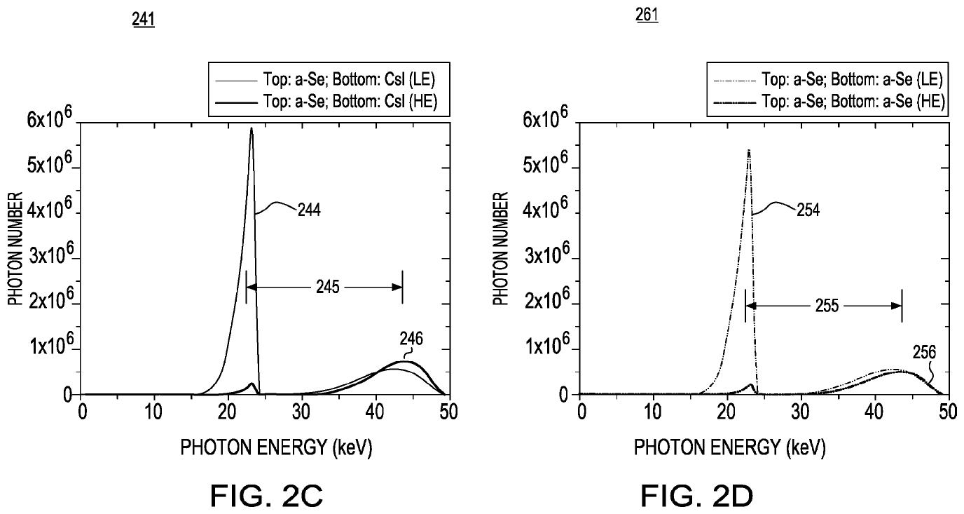

FIGS. 3A-3C illustrates the impact of filter thickness and thickness of the top detector material (T1) on spectral separation achieved for the dual-detector spectral imaging approach depicted embodiment of FIG. 1A.

FIG. 3A particularly illustrates a comparison 300 of the LE and HE spectral energy separation achieved for the single shot/dual layer detector approach of FIG. 1A using two different thicknesses of a Rhodium spectral separation filter 120, In particular, as shown in FIG. 3A, the single shot/dual layer detector approach using an Rh k-edge band filter of 200 μm thickness detects LE spectral radiation energy 304 and HE spectral energy 306 having respective spectral energy peaks separation 315 of about 20 keV resulting in improved LE and HE images. For comparison, FIG. 3A depicts the LE spectral radiation energy 305 and HE spectral radiation energy 307 achieved for the single shot/dual layer detector approach using an Rh k-edge band filter of 100 μm. As shown in FIG. 3A, approximately the same spectral energy separation is achieved in this single shot/dual layer detector approach.

FIG. 3B particularly illustrates a comparison 320 of the LE and HE spectral energy separation achieved for the single shot/dual layer detector approach of FIG. 1A using a front α-Se photoconductive detector 110 of two different thicknesses and a Rh spatial separation filter of 200 microns thick. In particular, as shown in FIG. 3B, the single shot/dual layer detector approach using a front α-Se photon integrating detector 110 of 200 μm thickness detects LE spectral radiation energy 314 and HE spectral energy 316 having respective spectral energy peaks separation 325 of about 20 keV resulting in improved LE and HE images. For comparison, FIG. 3B depicts the LE spectral radiation energy 317 and HE spectral radiation energy 319 achieved for the single shot/dual layer detector approach a front α-Se detector 110 of 150 μm thick. As shown in FIG. 3B, approximately the same spectral energy separation is achieved in this single shot/dual layer detector approach.

FIG. 3C illustrates the impact the provision of filter material located between front detector layer 110 and back detector layer 150 for the single shot/dual layer detector approach of FIG. 1A. FIG. 3C particularly shows a comparison 350 of respective plots of the Quantum detection efficiency measure of the detector as a function of the detected photon energy levels detected for different glass substrate thicknesses, e.g., the glass substrate(s) thickness of the front detector ranging between 300 microns-700 microns, with the optional addition of the glass substrate 352 from the bottom detector (when it is in a back-irradiation geometry). In comparison, an optional Cu filter (or other higher z materials) can be used.

FIG. 3D is a table 375 summarizing the resulting improvements in image quality as measured by a signal-difference-to-noise ratio (SDNR) 380 for different thicknesses of a front detector layer 110 and back detector layer 150 for the single shot/dual layer detector of FIG. 1A. In a first row, a single shot/dual layer detector having a front α-Se detector 110 of about 200 μm thickness and a back CsI photon integrating detector 150 of about 400 μm thickness and a Rh filter of about 200 μm thickness permits detection of a mean LE spectral radiation energy of 27.5 keV and a mean HE spectral energy of about 40.9 keV providing an improved SDNR of about 6.01 with an X-ray tube source loading of about 108.2 mAs. In the second row, a single shot/dual layer detector having a front α-Se detector 110 of about 200 μm thickness and a back CsI photon integrating detector 150 of about 400 μm thickness and a Rh filter of about 100 μm thickness permits detection of a mean LE spectral radiation energy of 28.0 keV and a mean HE spectral energy of about 39.6 keV providing an improved SDNR of about 5.22 with an X-ray tube source loading of about 18.2 mAs. Similarly, a single shot/dual layer detector having a front α-Se detector 110 of about 150 μm thickness and a back CsI photon integrating detector 150 of about 400 μm thickness and a Rh filter of about 200 μm thickness permits detection of a mean LE spectral radiation energy of 26.8 keV and a mean HE spectral energy of about 39.9 keV providing an improved SDNR of about 6.31 with an X-ray tube source loading of about 108.2 mAs.

Several embodiments of a dual-layer detector spectral imaging system approach of FIG. 1A for imaging an object 12 are provided with each embodiment particularly configured for digital breast tomosynthesis (DBT), i.e., 3D breast spectral imaging. The 3D spectral x-ray imaging system comprises various combinations of six components: 1) A spectral separation filter at the x-ray source output, comprising one or more materials with atomic numbers Z_{F1} to Z_{FN} and thicknesses T_{F1} to T_{FN}, to modulate the x-ray energy spectrum incident on the imaging subject; 2) A direct-conversion flat panel x-ray detector (“front detector”) that is first exposed to the x-ray beam transmitted through the imaging subject, having a lower atomic number Z₁, thickness T₁ and high spatial resolution; 3) A filter at the front detector's exit surface, comprising one or more materials with atomic number Z_{2F} to Z_{NF} and thicknesses T_{2F} to T_{2F}, which modulates the x-ray energy spectrum exiting the front detector; 4) A second, indirect-conversion flat panel x-ray detector (“back detector”) at the filter exit surface, having atomic number Z₃>Z₁ and thickness T₃; 5) A combined dual-layer detector with dual-shot x-ray exposure. This can be implemented with a switchable kVp and filter DBT system that provides up to four images for multiple material decomposition; and 6) a photon-counting detector (PCD) version of α-Se flat-panel imager (e.g., a field-Shaping multi-Well Avalanche Detector or SWAD) as the top (front) detector.

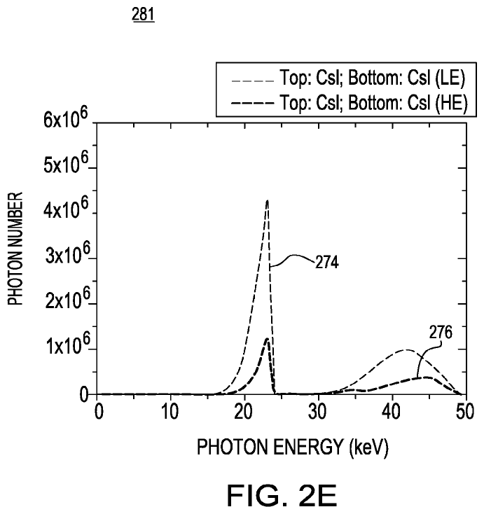

FIG. 4 shows a first embodiment of a spectral imaging system including a single shot/single layer detector 400. This first embodiment includes a combination of the spectral separation filter 120 located proximately in front of an X-ray radiation source 15 for absorbing a portion of X-ray radiation output of the radiation source 15. This spectral imaging system includes a single direct-conversion flat panel x-ray detector such as a photon counter detection (PCD) device 401 located on a substrate, e.g., glass substrate 225. As described in greater detail hereinbelow, the PCD 401 includes a flat panel imager such as an amorphous Selenium (α-Se) based field-Shaping multi-Well Avalanche Detector (SWAD) for photon counting at each pixel location. The α-Se x-ray photon counting flat-panel SWAD imager layer 401 converts received photons into an electrical pulse having a pulse height that is commensurate with the received photon's energy level. At each pixel location of the PCD layer, a semiconductor thresholding and count circuitry 230 detects the pulse height to distinguish either LE photons and HE photons received at the PCD layer from the incident radiation that is not absorbed by (i.e., transmitted through) the object 12. Threshold circuitry can include a first thresholding circuit for rejecting noise signals, and a second pulse thresholding circuit for use in comparing the height of the received pulses against a pulse height threshold. At a pixel, both LE and HE counter circuits count the number of photon interactions as electronic pulses received at that pixel location. Received photons from the radiation that exceed a pulse height threshold can be counted as HE photons and a HE storage “bin” maintains a photon number count value in a memory storage associated with that pixel location. Similarly, at the same pixel location, received photons from the radiation that are below a pulse height threshold can be counted as LE photons and a LE “bin” maintains a LE photon number count value in the memory storage associated with that pixel location.

As shown in FIG. 4, data acquisition circuits 98 including a programmed processor 99 and a memory configured to be in communication with each other, can receive the count values from each of the LE and HE bins and process these values to create a respective LE spectral image and HE spectral image of the object.

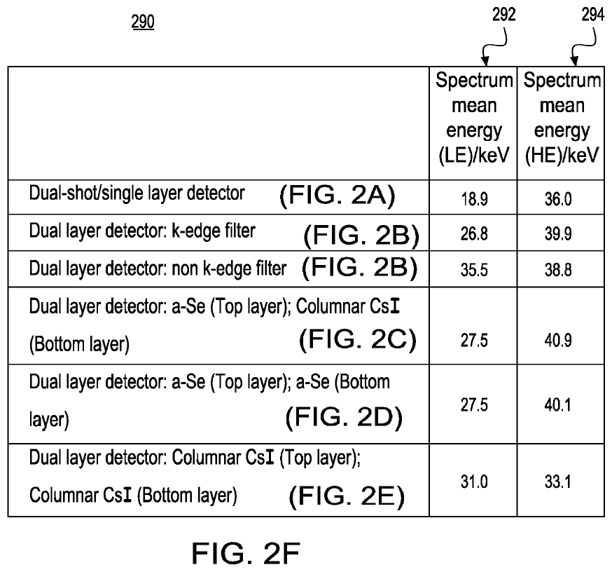

For embodiments employing a photon-counting detector (PCD) version of α-Se x-ray photon counting flat-panel imager (SWAD) as the top (front) detector, FIG. 5 depicts a cross-sectional schematic of a fully assembled SWAD device. In the cross-sectional schematic of the fully assembled SWAD device 500 of FIG. 5, there is depicted two distinct regions: (1) bulk region 510, formed by a thick film of α-Se for x-ray absorption and (2) a multi-well sensing region 525 composed of multiple wells 526 a plurality of pillars 550 deposited directly above the readout electronics, e.g., semiconductor pixel thresholding and CMOS counting circuitry 230 of FIG. 4. The sidewalls of the wells 526 are formed by dielectric pillars 550 with two grid electrodes 530, 535 embedded inside which can be utilized to create localized high fields within the well region for tunable avalanche gain and electrostatic shielding of the pixel electrode for fast unipolar time-differential charge sensing. When the grid electrodes 530, 535 are biased, the field lines above the wells bend, creating a field-shaping effect which guides drifting carriers into the wells. The strength of the electric field within the wells can be tuned to create a local high field region where carriers can undergo avalanche gain, while maintaining a low field just above the readout electronics and throughout the bulk. Additionally, the embedded grid electrodes act as Frisch grids, electrostatically shielding the pixel electrode from sensing any carrier motion within the bulk, while creating a strong near-field effect where collected signal rapidly increases as carriers enter the wells and undergo avalanche gain. The bulk region (˜200 μm) is an order of magnitude thicker than the well region to provide good x-ray absorption and minimize the effect of depth-dependent avalanche gain. A common high voltage electrode 560 is biased positively such that the fast carriers (holes, in α-Se) can be collected.

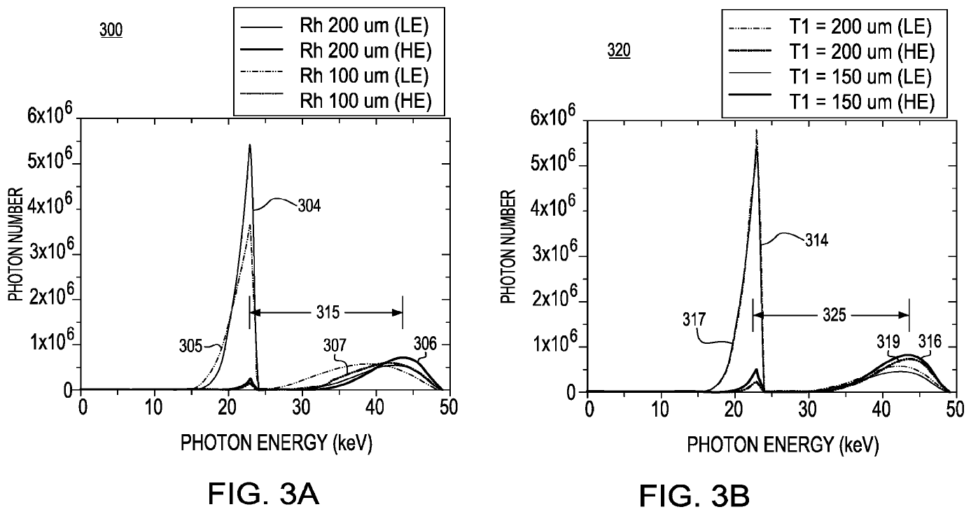

FIG. 6 depicts results 600 of a simulated pixel response of a photon counting SWAD device such as depicted in FIG. 5 with a 300 micron thick α-Se bulk layer exposed to a 49 kVp W spectrum filtered with 237 micron Cu and 4 cm thick breast with 50%/50% fibroglandular/adipose tissue composition. Pixel responses for both 75 micron and 100 micron pixel sizes are shown. The simulation results 600 are based on a multi-pixel geometry, whereby the pixel responses shown include the spectrum collected by a single central pixel with cross-talk from 8 neighboring pixels, incorporating the spatial energetic effects of charge sharing.

In an embodiment, use of a photon-counting version of α-Se x-ray photon counting flat-panel imager (SWAD) as the top detector provides a low-cost alternative to other photon counting detectors (PCD) using crystalline Cd(Zn)Te. Its energy resolution and count rate depends on the geometry of a Frische grid that is built on top of the CMOS photon counting integrated circuitry. With an avalanche gain of 10 and a linear Frische grid, a count rate of 100 k counts/second (cps) is possible, with energy resolution of 3 keV.

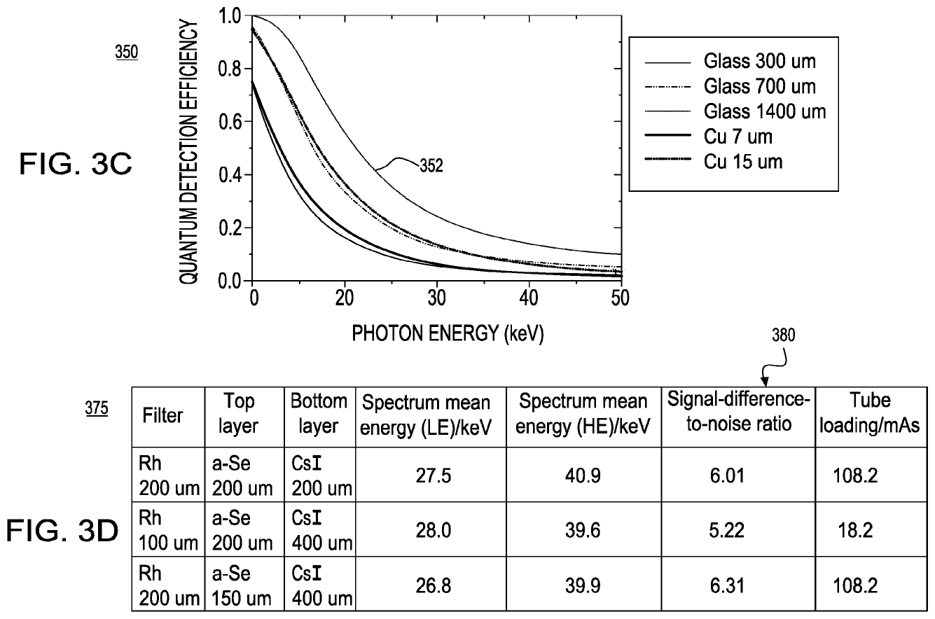

FIG. 7 depicts a second embodiment of a spectral imaging system including a single shot/dual layer detector 700. This embodiment includes the spectral separation filter 120 located proximately in front of an X-ray radiation tube source 15 for absorbing a portion of X-ray radiation output of the radiation source 15.

In an embodiment, spectral separation filter 120 at the x-ray source output, includes one or more materials, e.g., with atomic numbers Z_{F1} to Z_{FN} and thicknesses T_{F1} to T_{FN}, to modulate the x-ray energy spectrum 122 incident on the imaging subject 12. Filter materials are chosen with atomic numbers that selectively remove x-ray energies from the beam via preferential attenuation at energies near their K-edges, e.g., within 10 keV. Filter materials, thicknesses, and the order of their arrangement with respect to the x-ray source are chosen to shape the filter's energy transmission characteristics according to the initial energy spectrum, detector properties and spectral information of interest. For example, materials with K-edges near that of a breast imaging contrast material (e.g., iodine) can be selected. Examples separation filter materials can include Rh, Ag and Sn filter 120.

This spectral imaging system 700 includes a direct-conversion front photon counter detection (PCD) device 701 located on a substrate, e.g., glass substrate 225. As in the embodiment of FIG. 4, the PCD 701 includes a flat panel imager such as an amorphous Selenium (α-Se) based SWAD for photon counting at each pixel location. The α-Se flat-panel SWAD imager layer 701 converts received photons into an electrical pulse having a pulse height that is commensurate with the received photon's energy level. At each pixel location of the PCD layer, a thresholding and count circuitry 230 detects the pulse height to distinguish either LE photons and HE photons received at the PCD layer from the incident radiation that is not absorbed by (i.e., transmitted through) the object 12. Threshold circuitry can include a pulse thresholding circuit for use in comparing the height of the received pulses against a pulse height threshold value. At a pixel, both LE and HE counter circuits count the number of photon interactions as electronic pulses received at that pixel location. Received photons from the radiation that exceed a pulse height threshold can be counted as HE photons and a HE storage “bin” maintains a photon number count value in a memory storage associated with that pixel location. Similarly, at the same pixel location, received photons from the radiation that are below a pulse height threshold can be counted as LE photons and a LE “bin” maintains a LE photon number count value in the memory storage associated with that pixel location.

As shown in FIG. 7, data acquisition circuits 98 including a programmed processor 99 and a memory configured to be in communication with each other, that can receive the count values from each of the LE and HE bins and process these values to create a respective LE spectral image and HE spectral image of the object.

In an embodiment, the single shot/dual layer detector 700 further includes an indirect-conversion flat panel x-ray detector layer 751 formed on a second glass substrate 226 and attached underneath substrate 225 using a radiotransparent adhesive, for example. In an embodiment, the indirect-conversion flat panel x-ray detector layer 751 is an energy integrating detector (EID) such as columnar (col-) CsI. However, generally, the back EID detector 751 is of a material having an atomic number greater than the atomic number of the material of the first x-ray detector layer 701.

Included in the single-shot/dual layer detector 700 the back EID includes a scintillating phosphor layer (phosphor screen) for converting x-ray energy photons into light photons that can be sensed by an associated photosensor (photodetector) array circuitry 235 configured for indirectly capturing the energy of light photons from x-rays transmitted through the object. For example, the EID phosphor layer may include phosphor crystals that may capture the incident x-rays and convert the captured x-rays into light photons. Although not shown, a top surface of x-ray detector layer 751 can include a reflective layer, where the reflective layer may be made of a highly reflective material. For example, the reflective layer may be coated with a layer of white material, such as titanium dioxide. The reflective layer may reflect the scattered photons toward the photosensor array 235 in order for the photosensor array to capture any scattered photons. Thus, in some examples, incident x-rays may not be fully captured by the front detector (e.g., PCD layer) 701 to count all photon interactions. The uncaptured x-rays may pass through the PCD layer 701 and the crystals among the phosphor screen of EID layer 751 may convert the captured x-rays into light photons for detection.

In an embodiment, the back screen 751 may comprise a scintillating phosphor layer or material such as phosphor crystals that may capture the light photons. In some examples, the phosphor layer may be a powder or granular type (e.g., GdO2S2:Tb, CaWO4, BaFCl:Eu). In other examples, the screen phosphor may be comprised of nanometer-sized particles such as quantum dots, rather than the micron sized particles typical of “standard” screens such as GdO2S2:Tb. In still other examples, the scintillating material may be of the perovskite type. The back detector phosphor screen may emit light photons (e.g., photon bursts) in the visual light region.

The back detector phosphor screen may comprise a structured scintillating layer. For example, the back detector phosphor screen may include scintillating phosphor needle structures that may capture the light photons. In some examples, the back detector phosphor screen may be a vacuum deposited needle structure composed of CsI:Tl. In some examples, a combination of different types of scintillating materials and types may be used for the back screen.

The photosensor array 235 may include photosensitive storage elements and may include a plurality of switching elements (not shown). The second substrate 226 may be of small optical thickness, and in an alternative embodiment, may be disposed between the photosensor array 235 and the phosphor layer 751. The photosensitive storage elements and the switching elements may be disposed on top of the substrate 226. The photosensor array may be comprised of α-Si:H n-i-p photodiodes, MIS-type, or other types. The photosensor array may be sensitive to light incident the top side, and may have a low transmittance at the wavelengths emitted by the phosphor screen of EID layer 751. For example, the photosensor array 235 may have high optical absorption (above 90%) at the wavelength of the light emitted by the screens of layer 751 such that pixel crosstalk and crossover effects may be reduced. In an example, the substrate 226 may be of glass, plastic, or cellulose with thickness of 700 microns. The photosensor array 235 may capture the light photons and may convert the captured light photons into electrical signals, where the electrical signals may be used by a data acquisition electronics device (separate from the detector 700) to produce a digital image. For example, each switching element may correspond to a pixel of an image, such that toggling particular columns, rows, groups of pixels may cause a read out of a group of pixel values to produce an image.

In the system of FIG. 7, the single shot/dual layer detector 700 that combines the α-Se PCD front detector and an energy integrating CsI back detector, the thickness of α-Se is usually 200-300 microns. The transmitted photons from the α-Se PCD, which are primarily above 33 keV, can be integrated by the CsI detector, and added to the top energy bin of the α-Se PCD to ensure high quantum efficiency and SNR. In an embodiment, the number of photons in the high energy in by the SWAD PCD is approximately V3 that of the total detected photons by the SWAD and CsI EID together. The addition of CsI EID as the back detector provides substantial improvement in signal difference to noise ratio (SDNR) of the spectral images.

As shown in FIG. 7, data acquisition circuits 98 including a programmed processor 99 can receive the integrated HE photon energy values from each of the pixels and post-process these values to enhance the HE spectral image of the object.

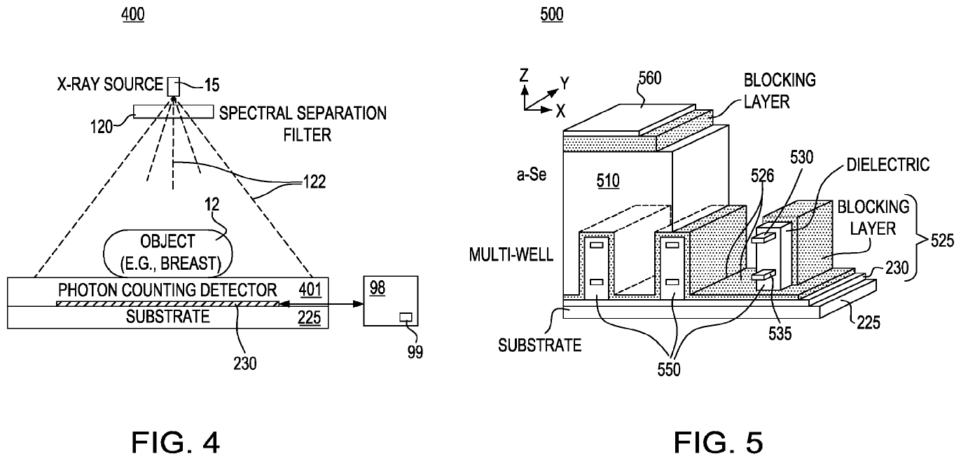

FIG. 8 depicts a further embodiment of a spectral imaging system including a single shot/dual layer detector 800. This embodiment optionally includes the spectral separation filter 120 or alternatively, includes an ordinary x-ray spectral imaging filter located proximately in front of an X-ray radiation tube source 15. As in the embodiment of FIG. 7, the spectral imaging system 800 includes a direct-conversion front photon counter detection (PCD) device 801 located on a substrate, e.g., glass substrate 225. The PCD 801 includes a flat panel imager such as an amorphous Selenium (α-Se) based SWAD for photon counting at each pixel location. The α-Se flat-panel SWAD imager layer 801 converts received photons into an electrical pulse having a pulse height that is commensurate with the received photon's energy level. At each pixel location of the PCD layer, a thresholding (binning) and count circuitry 230 that discriminates between LE photons and received HE photons and stores respective count values of detected LE and HE photons received at a pixel. Thresholding and count circuitry interfaces with data acquisition circuits 98 including a programmed processor 99 that receives the stored LE or HE photons at each pixel for generating respective LE and HE spectral images.

The single shot/dual layer detector 800 embodiment of FIG. 8 further includes an indirect-conversion flat panel x-ray detector layer 851 formed on a second substrate 226 and attached underneath substrate 225 using an adhesive, for example. In an embodiment, the indirect-conversion flat panel x-ray detector layer 851 is an energy integrating detector (EID) such as CsI. Generally, the back EID detector 851 is of a material having an atomic number greater than the atomic number of the material of the first x-ray detector layer 801.

As in the embodiment of FIG. 7, the addition of the CsI EID layer 851 as the back detector provides substantial improvement in signal difference to noise ratio (SDNR) of the spectral images as this layer captures the transmitted photons from the α-Se PCD, which are primarily above 33 keV, i.e., HE photons, that can be integrated by the CsI detector, and added to the top energy bin of the α-Se PCD to ensure high quantum efficiency and SNR. In particular, data acquisition circuits 98 including the programmed processor 99 can receive the integrated HE photon energy values from each of the pixels and post-process these values to enhance the HE spectral image of the object.

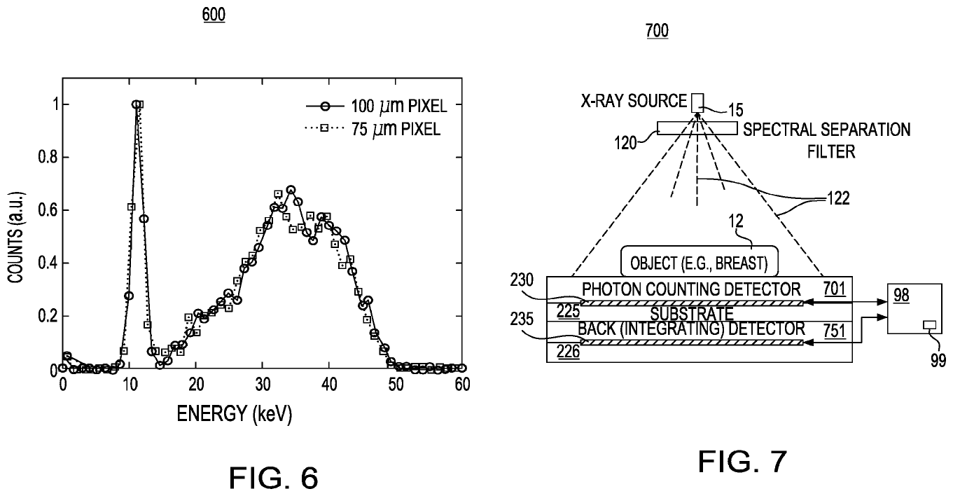

FIG. 9 depicts a further embodiment of a spectral imaging system including a single shot/dual layer detector 900. The embodiment of FIG. 9 includes a combination of the spectral separation filter 120 located proximately in front of an X-ray radiation source 15 for absorbing a portion of X-ray radiation output of the radiation source 15. This spectral imaging system includes a direct-conversion flat panel x-ray front detector 901 located on a substrate, e.g., glass substrate 225. Direct-conversion flat panel x-ray front detector 901 is first exposed to the x-ray beam transmitted through the imaging subject. In an embodiment, the direct-conversion flat panel x-ray detector layer 901 is an energy integrating detector (EID) including, but not limited to, a photoconductive material such as α-Se. In this embodiment, the α-Se EID flat panel x-ray detector layer 901 absorbs X-ray photons and excites electrons in the photoconductor to a conductive state in the material's conduction band. In the presence of an electric field, the electrons in the conduction band move along the electric field lines. Thus, charges released due to absorption of X-ray radiation can be collected by applying a potential to the across the α-Se EID material. Semiconductor pixel circuitry 930 located between the front imaging detector 901 and substrate. 225 can include a capacitor array or like electrical charge storage matrix for storing the collected charges released due to the absorption of X-ray radiation.

In an embodiment, front detector is of a material having an atomic number Z₁ and thickness T₁ and high spatial resolution chosen to preferentially absorb lower energy x-rays while transmitting higher energies, thereby allowing formation of a low energy image. Its high spatial resolution is leveraged to preserve image detail information, i.e., small structures and sharp edges. An amorphous selenium detector (Z₁=34, T₁=150 μm) is an example of the front detector material.

As further shown in FIG. 9, pixel circuitry 930 including the electrical charge storage matrix located between the front imaging detector 901 interfaces with data acquisition circuitry 98 including a programmed processor 99 and a memory for receiving the values associated with the stored LE or HE photons at each pixel and processing this data for generating respective LE and HE spectral images.

In this embodiment, located at an exit surface of the front detector, and shown sandwiched between the front integrating detector 901 and substrate 225 is a further spectral filter 940 for modulating the x-ray energy spectrum exiting the front detector 901. This filter 940 can comprise one or more materials with atomic number Z_{2F} to Z_{NF} and thicknesses ranging between T_{2F} to T_{2F}. In particular, material of filter 940 is chosen to attenuate low energy photons and facilitate device manufacture, e.g., glass used as a substrate for fabricating the front detector's active matrix. Thickness of filter 940 is tuned to a desired compromise between energy modulation and system sensitivity.

Further included is an indirect-conversion flat panel x-ray detector layer 951 formed on a second substrate 226 and attached underneath substrate 225 using an adhesive, for example. In an embodiment, the indirect-conversion flat panel x-ray detector layer 951 is an energy integrating detector (EID) of a material such as CsI. This second, indirect-conversion flat panel x-ray back detector can be of a material having an atomic number Z₃ greater than the atomic number Z₁ of the front detector material and is of a thickness T₃. In an embodiment, the back detector's atomic number is chosen to efficiently detect higher energy photons to form a high energy image. It may be matched to the K-edge of a contrast agent, e.g. CsI:Tl for iodine, to improve the agent's conspicuity in high energy images. The back detector's scintillator screen may be transparent or optically-turbid, structured or unstructured (e.g. columnar Cs:Tl or powder Gd₂O₂S:Tb), and comprise optically reflective or absorptive backings. The scintillator is coupled to a photodetector or photosensor array 235 by direct deposition, pressure contact, or in some embodiments by using a fiber optic plate to transmit the x-ray-induced light image to the photodetector without light spreading. The light sensors may be α-Si:H photodiodes, MIS-type, or other types known in the art. The thin film transistor (TFT) switching elements may be the α-Si:H type, a metal oxide (MOTFT) types, or other types known in the art.

As further shown in FIG. 9, data acquisition circuits 98 including the programmed processor 99 can receive the integrated HE photon energy values from each of the pixels and post-process these values to enhance the HE spectral image of the object.

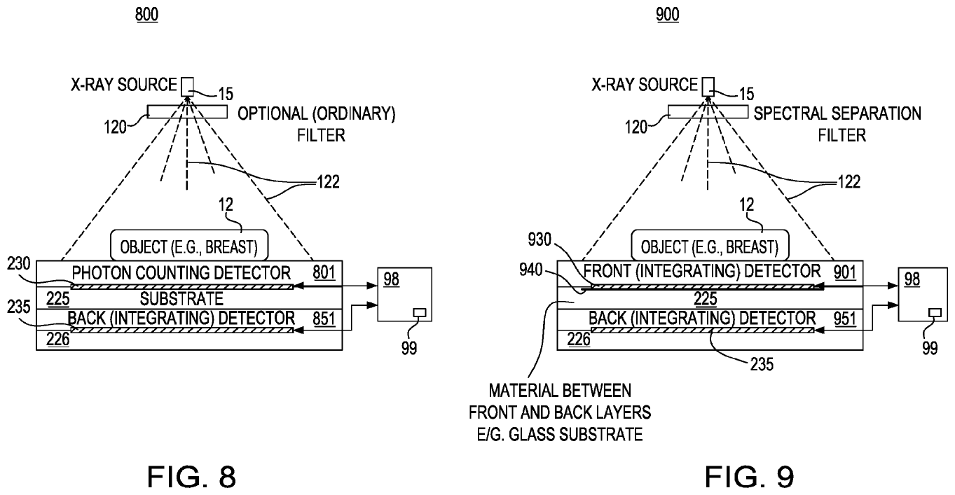

FIG. 10 depicts a further embodiment of a spectral imaging system including a dual shot exposure/dual layer detector 1000 with a switchable kVp and filter arrangement system. The embodiment of FIG. 10 includes a combination of a low energy spectral filter 124 located proximately in front of an X-ray radiation source 15 for providing a LE radiation portion, e.g., 28 keV of X-ray radiation output of the radiation source 15 during a first exposure and additionally includes a high energy spectral filter 126 located proximately in front of an X-ray radiation source 15 for providing a HE radiation portion, e.g., 49 keV of X-ray radiation output of the radiation source 15 during a second exposure.

The dual layer detector 1000 of FIG. 10 corresponds to the dual layer detector 900 of FIG. 9 and includes a direct-conversion flat panel x-ray front detector 1001 located on a substrate, e.g., glass substrate 225. Direct-conversion flat panel x-ray front detector 1001 is first exposed to the x-ray beam transmitted through the imaging object 12. In an embodiment, the direct-conversion flat panel x-ray detector layer 1001 is an energy integrating detector (EID) including, but not limited to, a photoconductive material such as α-Se. In this embodiment, the α-Se EID flat panel x-ray detector layer 1001 absorbs X-ray photons and excites electrons in the photoconductor to a conductive state in the material's conduction band. In the presence of an electric field, the electrons in the conduction band trove along the electric field lines. Thus, charges released due to absorption of X-ray radiation can be collected by applying a potential to the across the α-Se EID material. Semiconductor pixel circuitry 1030 located between the front imaging detector 1001 and substrate 225 can include a capacitor array or electrical charge storage matrix for storing the collected charges released due to the absorption of X-ray radiation.

In an embodiment, front detector is of a material having an atomic number Z₁ and thickness T₁ and high spatial resolution chosen to preferentially absorb lower energy x-rays while transmitting higher energies, thereby allowing formation of a low energy image. Its high spatial resolution is leveraged to preserve image detail information, i.e., small structures and sharp edges. An amorphous selenium detector (Z₁=34, T₁=150 μm) is an example of the front detector material.

As further shown in FIG. 10, pixel circuitry 1030 including the electrical charge storage matrix located between the front imaging detector 1001 interfaces with data acquisition circuitry 98 including a programmed processor 99 for receiving the values associated with the stored LE photons at each pixel and processing this data for generating LE spectral images.

In this embodiment, located at an exit surface of the front detector, and shown sandwiched between the front integrating detector 1001 and substrate 225 is a further spectral filter 1040 for modulating the x-ray energy spectrum exiting the front detector 901. This filter 1040 can comprise one or more materials with atomic number Z_{2F} to Z_{NF} and thicknesses ranging between T_{2F} to T_{2F}. In particular, material of filter 1040 is chosen to attenuate low energy photons and facilitate device manufacture, e.g., glass used as a substrate for fabricating the front detector's active matrix. Thickness of filter 1040 is tuned to a desired compromise between energy modulation and system sensitivity.

Further included is an indirect-conversion flat panel x-ray detector layer 1051 formed on a second substrate 226 and attached underneath substrate 225 using a radiotransparent adhesive, for example. In an embodiment, the indirect-conversion flat panel x-ray detector layer 1051 is an energy integrating detector (EID) of a material such as CsI. This second, indirect-conversion flat panel x-ray back detector can be of a material having an atomic number Z₃ greater than the atomic number Z₁ of the front detector material and is of a thickness T₃. In an embodiment, the back detector's atomic number is chosen to efficiently detect higher energy photons to form a high energy image. It may be matched to the K-edge of a contrast agent, e.g. CsI:Tl for iodine, to improve the agent's conspicuity in high energy images. The back detector's scintillator screen may be transparent or optically-turbid, structured or unstructured (e.g. columnar Cs:Tl or powder Gd₂O₂S:Tb), and comprise optically reflective or absorptive backings. The scintillator is coupled to a photodetector array 235 by direct deposition, pressure contact, or in some embodiments by using a fiber optic plate to transmit the x-ray-induced light image to the photodetector without light spreading. The light sensors may be α-Si:H photodiodes, MIS-type, or other types known in the art. The thin film transistor (TFT) switching elements may be the α-Si:H type, a metal oxide (MOTFT) types, or other types known in the art.

As further shown in FIG. 10, the data acquisition circuits 98 including the programmed processor 99 can receive the integrated HE photon energy values from each of the pixels and post-process these values to generate the HE spectral image of the object.

In an exemplary operation, in the dual-shot (dual-exposure) method, a first exposure is made with a low-energy beam and appropriate filter, e.g., 28 keV and Rh and a second exposure is made with a higher-energy beam and filter, e.g., 49 keV and Cu. Motion artifacts may be present but may be lessened by using fast keV switching and a rotatable filter wheel to register the appropriate filters in front of the radiation source in successive time instances. In the simplest case, the image data from the two detector layers are added to form the LE and HE images for use in dual-energy subtraction. That is, the results from both front and back detectors from the first (28 keV) exposure time are added to form the LE image, and the results from both detectors from the second (49 keV) exposure time are added to form the HE image. For the 28 keV exposure, the contribution of the CsI layer detector would be small because more of the absorption will occur in the front detector Se layer. For the 49 keV exposure the contribution of the CsI would be large because many of the higher energy x-rays will penetrate the Se layer. The benefits would include: (i) a greater energy separation in the LE and HE images, (ii) the LE image will appear very similar to a conventional mammogram done at 28 keV, and (iii) greater x-ray absorption of the HE beam than with Se alone due to the CsI layer. Further the four sets of image data could be useful for multiple material decomposition.

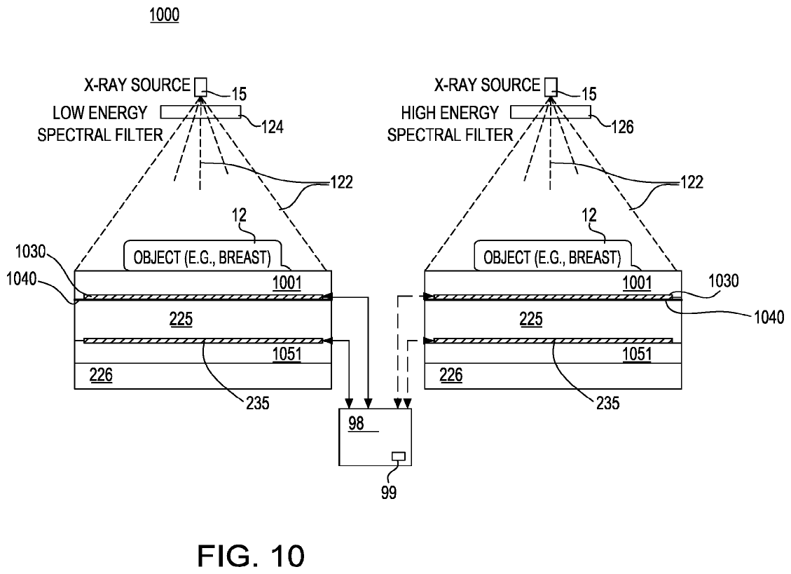

FIGS. 11A-11E depict simulation result images 1100 of a digital mammography, contrast enhanced dual energy mammography, digital breast tomosynthesis and contrast enhanced digital breast tomosynthesis. A top half of each image is from a digital breast phantom (with breast structure), and the bottom half is uniform breast tissue with 25% density. Iodine object sets, each arranged in a 4×4 arrays 1101 and 1102, are inserted to both upper and lower halves of the phantom. The iodine objects are spheres with diameters are 2, 3, 5, and 8 mm, and the iodine concentrations are 1, 2, 3, and 5 mg/ml respectively).

In the simulation images, FIG. 11A shows a simulated digital mammography image resulting from use of a single layer detector configured with a 50 μm thick Rh spatial separation filter (28 kVp) and a 300 μm thick α-Se detector.

FIG. 11B shows a simulated digital mammography image resulting from use of a single layer detector configured with a 237 μm thick Cu filter (49 kVp) and a 300 μm thick α-Se front detector.

FIG. 11C shows a contrast enhanced dual energy mammography image (e.g., dual layer detector of FIG. 9) configured with a 200 μm thick Rh spatial separation filter (49 kVp), 200 μm thick α-Se front detector, 700 μm thick glass, and a 400 μm thick columnar-CsI back detector.

FIG. 11D shows a contrast enhanced dual energy mammography image (e.g., dual layer detector of FIG. 9) configured with a 237 μm thick Cu spatial separation filter (49 kVp), 200 μm thick α-Se front detector, 700 μm thick glass substrate, and a 400 μm thick col-CsI back detector.

FIG. 11E shows a contrast enhanced dual energy mammography image (e.g., dual layer detector of FIG. 9) configured with a 200 μm thick Rh spatial separation filter (49 kVp), 200 μm thick col-CsI front detector, 700 μm thick glass substrate, and a 400 μm thick col-CsI back detector.

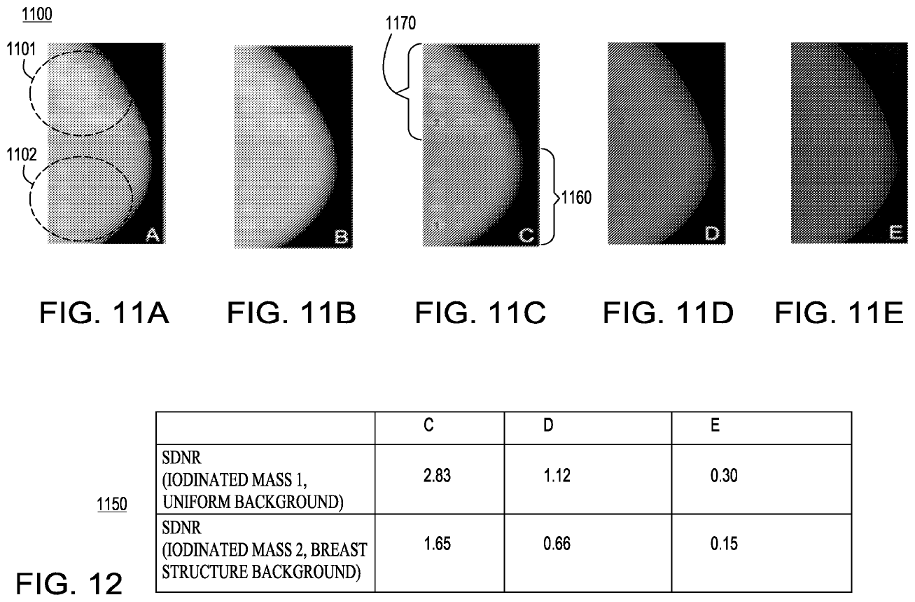

FIG. 12 shows a table 1150 depicting the Signal-Difference-to-Noise-Ratio in the simulated contrast enhanced dual energy mammography results of FIGS. 11C, 11D and 11E. As shown in table 1150, the best SDNR of 2.83 is achieved for the bottom half image portion, e.g., image half 1160 of FIG. 11C as compared to the SDNR of 1.65 achieved for the top image half 1170 as obtained using a simulated single-shot/dual layer detector of FIG. 9 configured with a 200 μm thick Rh spatial separation filter (49 kVp), 200 μm thick α-Se front detector, a 700 μm thick glass, and a 400 μm thick columnar-CsI back detector. Likewise, the SDNR values are increased for the bottom half image portions of FIGS. 11D and 11E as compared to the respective SDNR 65 achieved for their corresponding top image portions. As shown, embodiments of 11C, 11D implementing a dual-layer α-Se front detector and CsI back detector performs better than the dual layer embodiment using CsI front and back detectors.

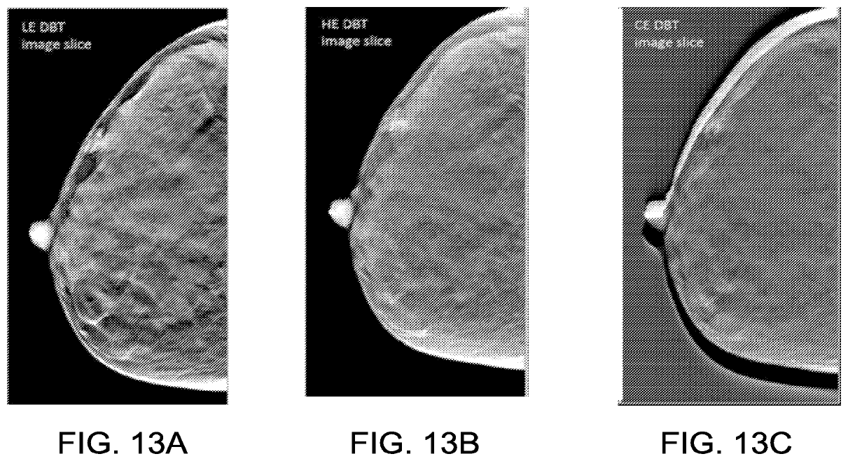

FIGS. 13A-13C depict images particularly showing the practical problem of DBT that the dual-layer detector approach of the embodiments of FIGS. 7 and 9 are intended to overcome. In FIGS. 13A-13C depicting respective LE DBT image slice, HE DBT image and CE DBT image slices, significant patient motion was observed between the HE and LE images and this problem would be exacerbated in DBT and CEDBT.

Embodiments of the system and method described herein overcome some of the shortcomings of various digital radiography systems and film-screen radiography systems by enabling a form of x-ray imaging which extracts material-specific information from a volume of interest, for example extracting the location and intensity of a contrast agent which has been injected into the body. Furthermore, this is done in a single exposure, eliminating the problem of patient motion between multiple exposures. Several ways of practicing the invention are disclosed, including the use of a dual-layer detector and also the use of a photon counting detector. The system and method enables acquisition of clinically valuable information like 3D breast tissue density, 3D microcalcification distribution and type, 3D distribution of contrast agent (e.g. iodine), and 3D material decomposition of breast lesions of interest (e.g. mass) in a digital breast tomography systems.

In mammography, spectral tomographic imaging offers more complete and accurate diagnostic information about the breast compared to conventional 2D full-field digital mammography (FFDM), spectral FFDM, and conventional DBT. This information includes 3D breast tissue density, 3D microcalcification distribution and type, 3D distribution of contrast agent (e.g. iodine), and 3D material decomposition of breast lesions of interest (e.g. mass).

Accordingly, the system and method herein provides for carrying out material-selective breast imaging, which enables the acquisition of clinically valuable information like the 3D location of a contrast agent, while eliminating image artifacts due to patient motion are disclosed.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements, if any, in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. An apparatus comprising:
a separation filter for spectrally separating radiation from an X-ray radiation source into a first energy level band and a second energy level band for incident radiation upon an imaging object;
a substrate;
an x-ray photon counting detector formed on the substrate, the x-ray photon counting detector comprising an array of detector pixels, each detector pixel comprising a sensor for detecting interactions of individual x-ray photons of the incident radiation transmitted through the imaging object during a fixed period of time; and
each detector pixel of the array having an associated count circuit operable to generate a first electrical signal representing a respective count of the number of detected interactions of individual x-ray photons of the first energy level band and a second electrical signal representing a respective count of the number of detected interactions of individual x-ray photons of the second energy level band, wherein the first electrical signals and second electrical signals from the detector pixels of the array provide respective energy spectral images of the imaging object.

2. The apparatus of claim 1, wherein each detector pixel sensor generates an electrical pulse having a height attribute commensurate with an energy level of the interacting x-ray photon, each associated count circuit of the array comprising a pulse height threshold discriminator circuit for incrementing a count of a detected interaction of an individual x-ray photon having a height attribute at or above certain threshold energy level used to discriminate between high and low energy levels.

3. The apparatus of claim 2, wherein radiation of the first energy level band is of a greater energy than radiation of the second energy level band, the certain threshold energy level corresponding to a pulse height attribute associated with a first energy level band.

4. The apparatus of claim 1, wherein the imaging object includes a contrast agent material having a characteristic K-edge atomic energy band level, the separation filter absorbing the X-ray radiation near the K-edge atomic energy band level.

5. The apparatus of claim 4, wherein the contrast agent is Iodine, the separation filter comprises a material selected from the group comprising Rh, Ag, Pd, In and Sn.

6. The apparatus of claim 4, wherein each detector pixel sensor comprises an amorphous Selenium (α-Se) based field shaping multi-well avalanche detector (SWAD).

7. The apparatus of claim 6, wherein the x-ray photon counting detector is a front detector formed upon the substrate, the apparatus further comprising:
a back detector located below the substrate, the back detector comprising:
a scintillating screen for converting incident radiation containing x-ray photons of the first energy level band transmitted through the imaging object and through the front detector into light photons; and
a photosensor array disposed between the scintillating screen and the substrate, the photosensor array operable to capture the light photons from the scintillating screen and convert the captured light photons into further electrical signals, the further electrical signals operable for combination with the first electrical signals from the detector pixel array to obtain images of the imaging object.

8. The apparatus of claim 7, wherein the back detector is an integrating detector, the scintillating screen of a material matching the characteristic K-edge atomic energy band level of the contrast agent material.

9. The apparatus of claim 7, wherein the scintillating screen is of a structured or columnar type or of an unstructured or granular type.

10. The apparatus of claim 7, wherein the back detector is a columnar CsI energy integrating detector.

11. The apparatus of claim 7, wherein the scintillating screen further comprises a backing, the backing comprising one of: a reflective surface or an absorptive surface.

12. The apparatus of claim 7, wherein the photosensor array comprises:
a plurality of photosensitive storage elements for capturing the at least a portion of the light photons from the scintillating screen; and
a plurality of switching elements where one switching element of the plurality of switching elements corresponds to one of the plurality of photosensitive storage elements, respectively, a transparent metal bias layer and a transparent 2D patterned metal layer, where the transparent 2D patterned metal layer faces the scintillating screen.

13. An apparatus comprising:
a separation filter for spectrally separating radiation from an X-ray radiation source into first energy level band and second energy level band for incident radiation upon an imaging object;
a first substrate;
a x-ray photon counting front detector formed on the first substrate, the x-ray photon counting detector comprising an array of detector pixels, each detector pixel comprising a sensor for detecting interactions of individual x-ray photons of the incident radiation transmitted through the imaging object during a fixed period of time;
each detector pixel of the array having an associated count circuit operable to generate a first electrical signal representing a respective count of the number of detected interactions of individual x-ray photons of the first energy level band and a second electrical signal representing a respective count of the number of detected interactions of individual x-ray photons of the second energy level band, wherein the first electrical signals and second electrical signals from the detector pixels of the array provide respective low energy and high energy spectral images of the imaging object; and
a back detector formed on a second substrate and located below said first substrate, said back detector comprising:
a scintillating screen for converting incident radiation containing x-ray photons of said second energy level band transmitted through the imaging object and through said front detector into light photons; and
a photosensor array disposed between said scintillating screen and the second substrate for said back detector, said photosensor array operable to capture the light photons from the scintillating screen and convert the captured light photons into further electrical signals, said further electrical signals operable for combination with said second electrical signals from said detector pixel array to obtain images of said imaging object.

14. The apparatus of claim 13, wherein each the front detector pixel sensor generates an electrical pulse having a height attribute commensurate with an energy level of the interacting x-ray photon, each associated count circuit of the array comprising a pulse height threshold discriminator circuit for incrementing a count of a detected interaction of an individual x-ray photon having a height attribute at or above certain threshold energy level.

15. The apparatus of claim 14, wherein radiation of the second energy level band is of a greater energy than radiation of the first energy level band, the certain threshold energy level corresponding to a pulse height attribute associated with a first energy level band.

16. The apparatus of claim 13, wherein the imaging object includes a contrast agent material having a characteristic K-edge atomic energy band level, the separation filter having an x-ray absorption edge for absorbing the X-ray radiation near the K-edge atomic energy band level.

17. The apparatus of claim 16, wherein the contrast agent is Iodine, the separation filter comprises a material selected from the group comprising Rh, Ag, Pd, In and Sn.

18. The apparatus of claim 13, wherein each front detector pixel sensor comprises an amorphous Selenium (α-Se) based field shaping multi-well avalanche detector (SWAD).

19. The apparatus of claim 16, wherein the back detector is an integrating detector, the scintillating screen of a material matching the characteristic K-edge atomic energy band level of the contrast agent material.

20. The apparatus of claim 19, wherein the scintillating screen is of a structured or columnar type or unstructured or granular type.

21. The apparatus of claim 18, wherein the back detector is a columnar CsI energy integrating detector.

22. The apparatus of claim 13, wherein the scintillating screen further comprises a backing, the backing comprising one of: a reflective surface or an absorptive surface.

23. The apparatus of claim 22, wherein the photosensor array comprises:
a plurality of photosensitive storage elements for capturing the at least a portion of the light photons from the scintillating screen; and
a plurality of switching elements where one switching element of the plurality of switching elements corresponds to one of the plurality of photosensitive storage elements, respectively, a metal bias layer and a 2D patterned metal layer, where the 2D patterned metal layer faces the scintillating screen.

24. An apparatus comprising:
a separation filter for spectrally separating radiation from an X-ray radiation source into a first energy level band and second energy level band for incident radiation upon an imaging object, the second energy level band being of a greater energy than the first energy level band;
a glass substrate;
a front integrating detector formed on the glass substrate, the front integrating detector comprising first pixel sensors for directly converting first energy level band x-ray photons of the incident radiation transmitted through the imaging into first image signals configurable to form a low energy image of the imaging object; and
a back integrating detector located below the glass substrate, the back integrating detector comprising second pixel sensors for indirectly converting second energy level band x-ray photons of the incident radiation transmitted through the imaging object and through the front integrating detector and the substrate and into second image signals configurable to form a high energy image of the imaging object.

25. The apparatus of claim 24, wherein the front integrating detector comprises:
a first photoconductive layer for converting incident radiation containing x-ray photons of the first energy level band transmitted through the imaging object into a charge; and
a first charge storage array disposed between the first photoconductive layer and the substrate for storing charges associated with the converted x-ray photons.

26. The apparatus of claim 25, wherein the back integrating detector comprises:
a scintillating screen for converting incident radiation containing x-ray photons of the second energy level band transmitted through the imaging object, the front integrating detector and the glass substrate into light photons; and
a photosensor array disposed between the scintillating screen and the glass substrate, the photosensor array operable to capture the light photons from the second scintillating screen and convert the captured light photons into the second imaging signals.

27. The apparatus of claim 26, wherein the imaging object includes a contrast agent material having a characteristic K-edge atomic energy band level, the separation filter having an x-ray absorption edge for absorbing the X-ray radiation near the K-edge atomic energy band level.

28. The apparatus of claim 27, wherein the scintillating screen is of a material matching the characteristic K-edge atomic energy band level of the contrast agent material.

29. The apparatus of claim 26, wherein the scintillating screen is of a structured or columnar type or unstructured or granular type.

30. The apparatus of claim 26, wherein the scintillating screen further comprises a backing, the backing comprising one of: a reflective surface or an absorptive surface.

31. The apparatus of claim 26, wherein the photosensor array comprises:
a plurality of photosensitive storage elements for capturing the at least a portion of the light photons from the scintillating screen; and
a plurality of switching elements where one switching element of the plurality of switching elements corresponds to one of the plurality of photosensitive storage elements, respectively, a metal bias layer and a transparent 2D patterned metal layer, where the transparent 2D patterned metal layer faces the scintillating screen.

32. The apparatus of claim 24, further comprising:
a filter disposed at an exit surface of the front detector, the filter comprising a material operable for modulating the X-ray radiation exiting the front detector and attenuating x-ray photons of the first energy level band.

33. The apparatus of claim 24, wherein the first photoconductive layer of the front energy integrating detector comprises an amorphous Selenium material and the scintillating screen of the back energy integrating detector comprises a CsI material.

34. An x-ray imaging system comprising:
an x-ray radiation source;
a first low energy band filter configured for registration in front of said x-ray radiation source for permitting transmission of a first low energy level band x-ray radiation for incidence upon an imaging object at a first time instance;
a second high energy band filter configured for subsequent registration in front of said x-ray radiation source for permitting transmission of a second high energy level band x-ray radiation for incidence upon said imaging object at a second time instance; and
a dual layer x-ray radiation detector comprising:
a glass substrate;
a front energy integrating detector formed on said glass substrate, said front energy integrating detector comprising first pixel sensors for directly converting photons of the first low energy level band of an incident x-ray radiation transmitted through an imaging object into first image signals during said first time instance and directly converting photons of the second higher energy level band of incident x-ray radiation transmitted through the imaging object into second image signals during said second time instance; and
a back energy integrating detector formed below said glass substrate, said back energy integrating detector comprising second pixel sensors for indirectly converting photons of the first low energy level band of the incident x-ray radiation transmitted through said imaging object and through said front energy integrating detector and said glass substrate into further first image signals during said first time instance, and indirectly converting photons of the second higher energy level band of incident x-ray radiation transmitted through the imaging object and through said front energy integrating detector and said glass substrate into further second image signals during said second time instance;
wherein said first image signals from the front detector are combinable with said further first image signals from the back detector to form a low energy image of said imaging object, and
said second image signals from the front detector are combinable with said further second image signals from the back detector to form a high energy image of said imaging object.

35. The system of claim 34, wherein said back energy integrating detector comprises:
a scintillating screen for converting incident radiation containing x-ray photons of the first low energy level band and the second higher energy level band transmitted through the imaging object and through the front energy integrating detector into light photons; and
a photosensor array disposed between the scintillating screen and the glass substrate, the photosensor array operable to capture the light photons from the scintillating screen and convert the captured light photons into respective said further first image signals and said further second image signals.

36. The system of claim 34, wherein said front energy integrating detector comprises:
a first photoconductive layer for converting incident radiation containing x-ray photons of the first low energy level band and second higher energy level band transmitted through the imaging object into respective charges; and
a charge storage array disposed between the first photoconductive layer and the substrate for storing the respective charges associated with the converted x-ray photons used to form respective said first image signals and said second image signals.

37. A method for x-ray imaging comprising:
providing a dual layer x-ray radiation detector below an object to be imaged with incident x-ray radiation, said dual layer x-ray radiation detector comprising:
a glass substrate;
a front energy integrating detector formed on said glass substrate comprising first pixel sensors; and
a back energy integrating detector formed below said glass substrate comprising second pixel sensors;
registering a first low energy band filter in front of an x-ray radiation source for permitting transmission of a first low energy level band x-ray radiation for incidence upon said imaging object at a first time instance;
registering a second high energy band filter in front of said x-ray radiation source for permitting transmission of a second high energy level band x-ray radiation for incidence upon said imaging object at a second time instance, and dual layer x-ray radiation detector receiving said first low energy level band x-ray radiation at said first time instance and second high energy level band x-ray radiation at said second time instance through said imaging object;
directly converting, using said first pixel sensors of said front energy integrating detector, photons of the first low energy level band of an incident x-ray radiation transmitted through the imaging object into first image signals during said first time instance and directly converting photons of the second higher energy level band of incident x-ray radiation transmitted through the imaging object into second image signals during said second time instance;
indirectly converting, using said second pixel sensors of said back energy integrating detector, photons of the first low energy level band of the incident x-ray radiation transmitted through said imaging object and through said front energy integrating detector and said glass substrate into further first image signals during said first time instance, and indirectly converting photons of the second higher energy level band of incident x-ray radiation transmitted through the imaging object and through said front energy integrating detector and said glass substrate into further second image signals during said second time instance;
forming, from said first image signals and further first image signals, a low energy image of said imaging object,
forming, from said second image signals and further second image signals, a high energy image of said imaging object.
